(19) United States
(12) Patent Application Publication
(10) Pub. No.: US 2010/0160756 A1
(43) Pub. Date: Jun. 24, 2010

(54) Membrane Layer for Electrochemical Biosensor and Method of Accommodating Electromagnetic and Radiofrequency Fields

(75) Inventors: James R. Petisce, San Clemente, CA (US); Henry W. Oviatt, Temecula, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/636,117

(22) Filed: Dec. 11, 2009

Related U.S. Application Data
(60) Provisional application No. 61/140,825, filed on Dec. 24, 2008.

(51) Int. Cl. A61B 5/1468; A61B 18/18

(52) U.S. Cl. 600/345; 606/33

(57) ABSTRACT

A method comprising providing an in vivo electrochemical biosensor, the biosensor comprising an electrode surface, a flux-limiting layer covering at least a portion of the electrode surface, covering at least a portion of the flux-limiting layer with a hydrophilic polymer membrane, and preventing or eliminating disruption of the output signal of the electrochemical biosensor by an external EMF or external RF source during in vivo use of the biosensor in a subject.

### CROSS-REFERENCE TO RELATED APPLICATIONS

This application claims the benefit of U.S. Provisional Application No. 61/140,825, filed Dec. 24, 2008, which is hereby incorporated by reference herein in its entirety.

### TECHNICAL FIELD

The present disclosure relates generally to preventing or eliminating electric and magnetic field (EMF) and/or radiofrequency field (RF) source disruptions of the output signal of an electrochemical analyte sensor. More particularly, the present disclosure relates to hydrophilic polymer membranes covering at least a portion of a flux-limiting layer of an electrochemical sensor.

### BACKGROUND

Among many problems impeding the development of a practical rapid and accurate amperometric sensor is a current need for the sensor technology to avoid external electromagnetic forces which would attenuate the output signal of the sensor. Attempts to reduce the effects of external electromagnetic forces in amperometric sensors have been addressed in a number of ways, for example by using separate and distinct electrical components and shielding, albeit with limited success. In certain cases, such as during a procedure involving an electrosurgical device, essentially no disruption of the output signal of the sensor would ideal. Unfortunately, the current amperometric sensors available on the market may not be capable of achieving the required protection in performance needed during specific medical procedures that involve concurrent use of electrosurgical devices producing or causing an EMF or RF disruption.

### SUMMARY

In general, electrochemical analyte sensors and sensor assemblies are disclosed that reduce or eliminate EMF or RF disruption when operated simultaneously with an electrosurgical device producing or causing an EMF or RF disruption. Such sensors are of particular use in more demanding sensing applications, such as monitoring during surgical procedures.

It is generally known that an amperometric device, such as a analyte sensor, having a polymeric, non-conducting outer coating, in some circumstances and in some environments, produces an electrostatic boundary layer about the flux-limiting layer when the sensor is biased. An external EMF or RF source generated concurrently with the operation of the biased sensor may cause a disruption of the electrostatic boundary and affect the performance of the sensor. Thus, it is envisaged that a hydrophilic polymer membrane positioned adjacent the outer coating of the sensor would reduce or eliminate the boundary layer disruption.

In one aspect, a method of reducing disruption of the output signal of the electrochemical biosensor by an external EMF or external RF source during in vivo use of the biosensor in a subject is provided. The method comprises providing an in vivo electrochemical biosensor, where the biosensor comprises an electrode surface and a flux-limiting layer covering at least a portion of the electrode surface, and covering at least a portion of the flux-limiting layer with a hydrophilic polymer membrane.

In another aspect, an electrochemical analyte sensor is provided. The sensor comprises an in vivo biosensor capable of sensing an analyte level in blood and outputting a signal corresponding to the analyte concentration. The in vivo biosensor comprises an electrode surface, an enzyme layer covering at least a portion of the electrode surface, a flux-limiting layer covering at least a portion of the enzyme layer and at least a portion of the electrode surface, and a hydrophilic polymer membrane covering at least a portion of the flux-limiting layer. Disruption of the output signal of the analyte sensor, when operated in the presence of an external EMF or external RF source during in vivo use, is prevented or eliminated.

In one aspect, the external EMF or external RF source is generated by an electrosurgical unit (ESU).

In one aspect, the electrosurgical unit operates at a frequency between about 350 KHz and about 4 MHz.

In one aspect, the hydrophilic polymer membrane accelerates reformation of a boundary layer comprising charged species about the flux-limiting layer of the electrochemical biosensor during in vivo use thereof in a subject.

In one aspect, the hydrophilic polymer membrane comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyvinyl acetate, polyelectrolytes and copolymers thereof.

In one aspect, the hydrophilic polymer membrane is covalently or ionically coupled to the flux-limiting layer.

In one aspect, the electrochemical sensor further comprises an interference layer at least partially covering the electrode surface.

In one aspect, the electrochemical sensor further comprises an hydrophilic layer at least partially covering the electrode surface.

In one aspect, the interference layer of the electrochemical sensor comprises a cellulosic derivative.

In one aspect, the interference layer of the electrochemical sensor is cellulose acetate butyrate.

In one aspect, the electrochemical sensor further comprises an enzyme layer at least partially covering the interference layer.

In one aspect, the electrochemical sensor enzyme layer comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyelectrolyte and copolymers thereof.

In one aspect, the electrochemical sensor enzyme layer comprises an enzyme and poly-N-vinylpyrrolidone.

In one aspect, the electrochemical sensor enzyme layer comprises glucose oxidase, poly-N-vinylpyrrolidone, and an amount of crosslinking agent sufficient to immobilize the glucose oxidase.

In one aspect, the flux-limiting layer of the sensor comprises a polymer selected from polysilicones, polyurethanes and copolymers or blends thereof.

In one aspect, the flux-limiting layer of the sensor comprises a vinyl polymer.

In one aspect, the flux-limiting layer of the sensor comprises vinyl acetate monomeric units.

In one aspect, the flux-limiting layer of the sensor is poly(ethylene-vinylacetate).

### BRIEF DESCRIPTION OF THE DRAWINGS

FIG. 1 shows an amperometric sensor coupled to a flex circuit having a working electrode according to an embodiment of the invention.

FIG. 2 is a side cross-sectional view of a working electrode portion of the sensor of shown prior to application of a hydrophilic polymer membrane according to an embodiment of the invention.

FIG. 3 is a side cross-sectional view of the working electrode portion of the sensor as in FIG. 2, shown after application of the hydrophilic polymer membrane according to an embodiment of the invention.

FIG. 4 is a side view of a multi-lumen catheter with a sensor assembly according to an embodiment of the invention.

FIG. 5 is a detail of the distal end of the multi-lumen catheter of FIG. 4 according to an embodiment of the invention.

### DETAILED DESCRIPTION

Typically, an electrochemical sensor is configured with layers, each layer having at least one function associated with the detection of a target analyte. For example, an electrochemical analyte sensor may include an outermost layer for controlling the flux of one or more species to the electroactive surface of the sensor. The outmost layer of an in vivo sensor typically is hydrophobic and essentially is or function as a flux-limiting layer. During the normal in vivo use of a biased electrochemical analyte sensor that comprises a flux-limiting layer, an electrostatic boundary layer is formed around the flux-limiting layer. The boundary layer is comprised, at least in part, of charged species. While not to be held to any particular theory, it is generally believed that exposure of the flux-limiting layer of the biased electrochemical analyte sensor to an external EMF or RF source causes disruption of this boundary layer and as a result, disrupts the output signal of the sensor. For example, during exposure to the EMF or RF source the output signal may spike and/or plateau at a higher output level than before exposure to the EMF or RF source. Moreover, the output signal of the sensor may not return to its pre-EMF exposed or pre-RF exposed level, potentially rendering the sensor inoperable, un-calibrated, and/or unreliable. Accordingly, in some circumstances, it is generally believed that a hydrophilic polymer membrane should be employed adjacent the outer coating of the sensor. Alternatively, in some circumstances, it is generally believed that a hydrophilic polymer membrane should be coupled to the outer coating of a sensor. Applicants have surprisingly reasoned and believe that the embodiments disclosed herein will substantially eliminate or reduce EMF or RF disruption of the sensor output signal when operated simultaneously with exposure to an EMF or RF source. Herein disclosed and described is an electrochemical analyte sensor and a method of reducing or eliminating the disruption of the output signal of an electrochemical analyte sensor during exposure to an EMF or RF source, for example, produced by an electrosurgical unit (ESU).

The following description and examples illustrate some exemplary embodiments of the disclosed invention in detail. Those of skill in the art will recognize that there may be numerous variations and modifications of this invention that may be encompassed by its scope. Accordingly, the description of a certain exemplary embodiment is not intended to limit the scope of the present invention.

### DEFINITIONS

In order to facilitate an understanding of the various aspects of the embodiments disclosed herein, the following are defined below.

The term “analyte” as used herein refers without limitation to a substance or chemical constituent of interest in a biological fluid (for example, blood) that may be analyzed. The analyte may be naturally present in the biological fluid, the analyte may be introduced into the body, or the analyte may be a metabolic product of a substance of interest or an enzymatically produced chemical reactant or chemical product of a substance of interest. Preferably, analytes include chemical entities capable of reacting with at least one enzyme and quantitatively yielding an electrochemically reactive product that is either amperiometrically or voltammetrically detectable.

The phrases and terms “analyte measuring device,” “sensor,” and “sensor assembly” as used herein refer without limitation to an area of an analyte-monitoring device that enables the detection of at least one analyte. For example, the sensor may comprise a non-conductive portion, at least one working electrode, a reference electrode, and a counter electrode (optional), forming an electrochemically reactive surface at one location on the non-conductive portion and an electronic connection at another location on the non-conductive portion, and a one or more layers over the electrochemically reactive surface.

The term “bipolar” as used herein, refers without limitation to electrical surgical units having two electrode surfaces contained within the surgical instrument. For example, a bipolar electrical surgical unit comprises a surgical instrument where the current flow is generally confined to the space between the two electrode surfaces of the surgical instrument and a dispersive or ground pad is not employed.

The term “break-in” as used herein refers without limitation to a time duration, after sensor deployment, where an electrical output from the sensor achieves a substantially constant value following contact of the sensor with a solution. Break-in is inclusive of configuring the sensor electronics by applying different voltage settings, starting with a higher voltage setting and then reducing the voltage setting and/or pre-treating the operating electrode with a negative electric current at a constant current density. Break-in is inclusive of chemical/electrical equilibrium of one or more of the sensor components such as membranes, layers, enzymes and electronics, and may occur prior to calibration of the sensor output. For example, following a potential input to the sensor, an immediate break-in would be a substantially constant current output from the sensor. By way of example, an immediate break-in for a glucose electrochemical sensor after contact with a solution, would be a current output representative of +/−5 mg/dL of a calibrated glucose concentration within about thirty minutes or less after deployment. The term “break-in” is well documented and is appreciated by one skilled in the art of electrochemical glucose sensors, however it may be exemplified for a glucose sensor, as the time at which reference glucose data (e.g., from an SMBG meter) is within +/−5 mg/dL of the measured glucose sensor data.

The phrase “capable of” as used herein, when referring to recitation of function associated with a recited structure, is inclusive of all conditions where the recited structure can actually perform the recited function. For example, the phrase “capable of” includes performance of the function under normal operating conditions, experimental conditions or laboratory conditions, as well as conditions that may not or can not occur during normal operation.

The term “cellulose acetate butyrate” as used herein refers without limitation to compounds obtained by contacting cellulose with acetic anhydride and butyric anhydride.

The term “comprising” and its grammatical equivalents, as used herein is synonymous with “including,” “containing,” or “characterized by,” and is inclusive or open-ended and does not exclude additional, unrecited elements or method steps.

The phrases “continuous analyte sensing” and “continual analyte sensing” (and the grammatical equivalents “continuously” and continually“) as used herein refer without limitation to a period of analyte concentration monitoring that is continuously, continually, and/or intermittently (but regularly) performed.

The phrase “continuous glucose sensing” as used herein refers without limitation to a period of glucose concentration monitoring that is continuously, continually, and/or intermittently (but regularly) performed. The period may, for example, at time intervals ranging from fractions of a second up to, for example, 1, 2, or 5 minutes, or longer.

The term “cover” and its grammatical equivalents is used herein refers without limitation to its normal dictionary definition. The term cover is inclusive of one or more intervening layers. For example, a flux-limiting layer covering at least a portion of an electrode is inclusive of one or more intervening layers between the flux-limiting layer and the electrode.

The terms “crosslink” and “crosslinking” as used herein refer without limitation to joining (e.g., adjacent chains of a polymer and/or protein) by creating covalent or ionic bonds. Crosslinking may be accomplished by known techniques, for example, thermal reaction, chemical reaction or ionizing radiation (for example, electron beam radiation, UV radiation, X-ray, or gamma radiation). For example, reaction of a dialdehyde such as glutaraldehyde with a hydrophilic polymer-enzyme composition would result in chemical crosslinking of the enzyme and/or hydrophilic polymer.

The term “disruption of the output current” as used herein generally refers to any external field effecting the electrochemical sensor signal output. External field effects include, for example, spiking of signal and/or a plateau of signal. Disruption of the output current may be caused by one or more electromagnetic fields produced by an alternating current. An example includes an electrosurgical unit (ESU), which can generate an AC-based electromagnetic field (EMF) that will typically range from about 0.2 mG to several hundred mG in strength. Another example would include, for example, conducted interference originated from the coupling of ambient radiated interference or capacitively, inductively or galvanically induced interference by an emitting radiofrequency (RF) source, typically at audio and lower radio frequencies. Such RF sources may cause a disruption in the output signal of the sensor when the field strength exceeds about 1 to 3 V/m, but lesser field strengths may also cause a disruption. An ESU may generate a RF source capable of disrupting the output signal of an in vivo sensor.

The phrase “electroactive surface” as used herein is refers without limitation to a surface of an electrode where an electrochemical reaction takes place. For example, at a predetermined potential, H₂O₂ reacts with the electroactive surface of a working electrode to produce two protons (2H+), two electrons (2e⁻) and one molecule of oxygen (O₂), for which the electrons produce a detectable electronic current. The electroactive surface may include on at least a portion thereof, a chemically or covalently bonded adhesion promoting agent, such as aminoalkylsilane, and the like.

The phrase “electrosurgical unit” or “ESU” as used herein interchangeably and generally refers without limitation to a medical device capable of surgically interacting with tissue using high frequency generated electrical energy. For example, an ESU may use frequencies of between about 350 KHz to about 4 MHz or more. An ESU may use a RF generator operating in the range of about 80 W to about 500 W. ESU's include devices capable of sensing resistance of tissue and/or adjusting voltage and/or current during use. An example of an ESU is a Bovie unit.

The phrase “enzyme layer” as used herein refers without limitation to a permeable or semi-permeable membrane comprising one or more domains that may be permeable to reactants and/or co-reactants employed in determining the analyte of interest. As an example, an enzyme layer comprises an immobilized glucose oxidase enzyme in a hydrophilic polymer, which catalyzes an electrochemical reaction with glucose and oxygen to permit measurement of a concentration of glucose.

The term “flux limiting membrane” as used herein refers to a semi-permeable membrane that controls the flux of one or more analytes to the underlying enzyme layer. By way of example, for a glucose sensor, the flux limiting membrane preferably renders oxygen in a non-rate-limiting excess. As a result, the upper limit of linearity of glucose measurement is extended to a much higher value than that which is achieved without the flux limiting membrane. The flux limiting membrane may be an electrically insulating material, for example, a material with a dielectric constant of less than about 12.

The terms “interferants,” “interferents” and “interfering species,” as used herein refer without limitation to effects and/or species that otherwise interfere with a measurement of an analyte of interest in a sensor to produce a signal that does not accurately represent the analyte measurement. For example, in an electrochemical sensor, interfering species may be compounds with oxidation potentials that substantially overlap the oxidation potential of the analyte to be measured.

The terms “monopolar” or “unipolar” as used herein are used interchangeably and refer without limitation, to electrical surgical units having only one electrode surface contained within the surgical instrument. For example, a monopolar electrical surgical unit comprises a surgical instrument having an electrode surface and an external dispersive or “ground” pad.

The term “polyelectrolyte” as used herein refers to a high molecular weight material having pendent ionizable groups. The molecular weight of polyelectrolytes may range from a few thousand to millions of Daltons. In one aspect, polyelectrolytes are exclusive of polymers with terminal ionizable groups and essentially no pendent ionizable groups, for example, Nafion.

The term “subject” as used herein refers without limitation to mammals, particularly humans and domesticated animals.

The phrase “vinyl ester monomeric units” as used herein refers to compounds and compositions of matter which are formed from the polymerization of an unsaturated monomer having ester functionality. For example, polyethylene vinyl acetate polymer and copolymers thereof are compounds comprising vinyl ester monomeric units.

### Sensor System and Sensor Assembly

The aspects herein disclosed relate to the use of an analyte sensor system that measures a concentration of analyte of interest or a substance indicative of the concentration or presence of the analyte capable of functioning in the presence of an external EMF or RF source. The sensor system is a continuous device, and may be used, for example, as or part of a subcutaneous, transdermal (e.g., transcutaneous), or intravascular device. The analyte sensor may use an enzymatic, chemical, electrochemical, or combination of such methods for analyte-sensing. The output signal is typically a raw signal that is used to provide a useful value of the analyte of interest to a user, such as a patient or physician, who may be using the device. Accordingly, appropriate smoothing, calibration, and evaluation methods may be applied to the raw signal.

Generally, the sensor comprises at least a portion of the exposed electroactive surface of a working electrode surrounded by a plurality of layers. Preferably, an interference layer is deposited over and in contact with at least a portion of the electroactive surfaces of the sensor (working electrode and optionally the reference electrode) to provide protection of the exposed electrode surface from the biological environment and/or limit or block of interferents. An enzyme layer is deposited over and in contact with at least a portion of the interference layer. In one aspect, the interference layer and enzyme layer provides for rapid response and stabilization of the signal output of the sensor and/or eliminates the need to pre-treat the electroactive surface of the electrode with fugitive species, such as salts and electrolyte layers or domains, which simplifies manufacture and reduces lot-to-lot variability of the disclosed sensors. A flux-limiting layer is deposited over the enzyme layer and/or the sensor assembly to control the flux of analyte or co-analytes to the enzyme layer. A hydrophilic polymer membrane is applied over the flux-limiting layer to eliminate or reduce disruption of the output signal of the sensor when used in the presence of an EMF or RF source.

One exemplary embodiment described in detail below utilizes a medical device, such as a catheter, with a glucose sensor assembly. In one aspect, a medical device with an analyte sensor assembly is provided for inserting the into a subject's vascular system. The medical device with the analyte sensor assembly may include associated therewith an electronics unit associated with the sensor, and a receiver for receiving and/or processing sensor data. Although a few exemplary embodiments of continuous glucose sensors may be illustrated and described herein, it should be understood that the disclosed embodiments may be applicable to any device capable of substantially continual or substantially continuous measurement of a concentration of analyte of interest and for providing an rapid and accurate output signal that is representative of the concentration of that analyte.

### Electrode and Electroactive Surface

The electrode and/or the electroactive surface of the sensor or sensor assembly disclosed herein comprises a conductive material, such as platinum, platinum-iridium, palladium, graphite, gold, carbon, conductive polymer, alloys, ink or the like. Although the electrodes can by formed by a variety of manufacturing techniques (bulk metal processing, deposition of metal onto a substrate, or the like), it may be advantageous to form the electrodes from screen printing techniques using conductive and/or catalyzed inks. The conductive inks may be catalyzed with noble metals such as platinum and/or palladium.

In one aspect, the electrodes and/or the electroactive surfaces of the sensor or sensor assembly are formed on a flexible substrate, such as a flex circuit. In one aspect, a flex circuit is part of the sensor and comprises a substrate, conductive traces, and electrodes. The traces and electrodes may be masked and imaged onto the substrate, for example, using screen printing or ink deposition techniques. The trace and the electrodes, and the electroactive surface of the electrode may be comprised of a conductive material, such as platinum, platinum-iridium, palladium, graphite, gold, carbon, conductive polymer, alloys, ink or the like.

In one aspect, a counter electrode is provided to balance the current generated by the species being measured at the working electrode. In the case of a glucose oxidase based glucose sensor, the species being measured at the working electrode is H₂O₂. Glucose oxidase catalyzes the conversion of oxygen and glucose to hydrogen peroxide and gluconate according to the following reaction: Glucose+O₂→Gluconate+H₂O₂. Oxidation of H₂O₂ by the working electrode is balanced by reduction of any oxygen present, or other reducible species at the counter electrode. The H₂O₂ produced from the glucose oxidase reaction reacts at the surface of working electrode and produces two protons (2H⁺), two electrons (2e⁻), and one oxygen molecule (O₂).

In one aspect, additional electrodes may be included within the sensor or sensor assembly, for example, a three-electrode system (working, reference, and counter electrodes) and/or one or more additional working electrodes configured as a baseline subtracting electrode, or which is configured for measuring additional analytes. The two working electrodes may be positioned in close proximity to each other, and in close proximity to the reference electrode. For example, a multiple electrode system may be configured wherein a first working electrode is configured to measure a first signal comprising glucose and baseline and an additional working electrode substantially similar to the first working electrode without an enzyme disposed thereon is configured to measure a baseline signal consisting of baseline only. In this way, the baseline signal generated by the additional electrode may be subtracted from the signal of the first working electrode to produce a glucose-only signal substantially free of baseline fluctuations and/or electrochemically active interfering species.

In one aspect, the sensor comprises from 2 to 4 electrodes. The electrodes may include, for example, the counter electrode (CE), working electrode (WE1), reference electrode (RE) and optionally a second working electrode (WE2). In one aspect, the sensor will have at least a CE and WE1. In one aspect, the addition of a WE2 is used, which may further improve the accuracy of the sensor measurement. In one aspect, the addition of a second counter electrode (CE2) may be used, which may further improve the accuracy of the sensor measurement.

The electroactive surface may be treated prior to application of any of the subsequent layers. Surface treatments may include for example, chemical, plasma or laser treatment of at least a portion of the electroactive surface. By way of example, the electrodes may be chemically or covalently contacted with one or more adhesion promoting agents. Adhesion promoting agents may include for example, aminoalkylalkoxylsilanes, epoxyalkylalkoxylsilanes and the like. For examples, one or more of the electrodes may be chemically or covalently contacted with a solution containing 3-glycidoxypropyltrimethoxysilane.

In some alternative embodiments, the exposed surface area of the working (and/or other) electrode may be increased by altering the cross-section of the electrode itself. Increasing the surface area of the working electrode may be advantageous in providing an increased signal responsive to the analyte concentration, which in turn may be helpful in improving the signal-to-noise ratio, for example. The cross-section of the working electrode may be defined by any regular or irregular, circular or non-circular configuration.

### Hydrophilic Layer

In one aspect, the electrochemical sensor comprises a hydrophilic layer over the electrode/electroactive surface and/or in direct contact with the electrode/electroactive surface. The hydrophilic layer may be formed from poly-N-vinylpyrrolidone (PVP), poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polymers with pendent ionizable groups, and copolymers or blends thereof. Preferably, the hydrophilic layer comprises poly-N-vinylpyrrolidone or polyelectrolytes.

### Interference Layer

Interferents may be molecules or other species that may be reduced or oxidized at the electrochemically reactive surfaces of the sensor, either directly or via an electron transfer agent, to produce a false positive analyte signal (e.g., a non-analyte-related signal). This false positive signal generally causes the subject's analyte concentration to appear higher than the true analyte concentration. For example, in a hypoglycemic situation, where the subject has ingested an interferent (e.g., acetaminophen), the artificially high glucose signal may lead the subject or health care provider to believe that they are euglycemic or, in some cases, hyperglycemic. As a result, the subject or health care provider may make inappropriate or incorrect treatment decisions.

In one aspect, an interference layer is provided on the sensor or sensor assembly that substantially restricts or eliminates the passage there through of one or more interfering species. Interfering species for a glucose sensor include, for example, acetaminophen, ascorbic acid, bilirubin, cholesterol, creatinine, dopamine, ephedrine, ibuprofen, L-dopa, methyl dopa, salicylate, tetracycline, tolazamide, tolbutamide, triglycerides, urea and uric acid. The interference layer may be less permeable to one or more of the interfering species than to a target analyte species.

In an embodiment, the interference layer is formed from one or more cellulosic derivatives. In one aspect, mixed ester cellulosic derivatives may be used, for example, cellulose acetate butyrate, cellulose acetate phthalate, cellulose acetate propionate, cellulose acetate trimellitate, as well as their copolymers and terpolymers, with other cellulosic or non-cellulosic monomers, including cross-linked variations of the above. Other polymers, such as polymeric polysaccharides having similar properties to cellulosic derivatives, may be used as an interference material or in combination with the above cellulosic derivatives. Other esters of cellulose may be blended with the mixed ester cellulosic derivatives.

In one aspect, the interference layer is formed from cellulose acetate butyrate. Cellulose acetate butyrate is a cellulosic polymer having both acetyl and butyl groups, and hydroxyl groups. A cellulose acetate butyrate having about 35% or less acetyl groups, about 10% to about 25% butyryl groups, and hydroxyl groups making up the remainder may be used. A cellulose acetate butyrate having from about 25% to about 34% acetyl groups and from about 15 to about 20% butyryl groups may also be used, however, other amounts of acetyl and butyryl groups may be used. A preferred cellulose acetate butyrate contains from about 28% to about 30% acetyl groups and from about 16 to about 18% butyryl groups.

Cellulose acetate butyrate with a molecular weight of about 10,000 daltons to about 75,000 daltons is preferred, preferably from about 15,000, 20,000, or 25,000 daltons to about 50,000, 55,000, 60,000, 65,000, or 70,000 daltons, and more preferably about 65,000 daltons is employed. In certain embodiments, however, higher or lower molecular weights may be used or a blend of two or more cellulose acetate butyrates having different molecular weights may be used.

A plurality of layers of cellulose acetate butyrate may be combined to form the interference layer in some embodiments, for example, two or more layers may be employed. It may be desirable to employ a mixture of cellulose acetate butyrates with different molecular weights in a single solution, or to deposit multiple layers of cellulose acetate butyrate from different solutions comprising cellulose acetate butyrate of different molecular weights, different concentrations, and/or different chemistries (e.g., wt % functional groups). Additional substances in the casting solutions or dispersions may be used, e.g., casting aids, defoamers, surface tension modifiers, functionalizing agents, crosslinking agents, other polymeric substances, substances capable of modifying the hydrophilicity/hydrophobicity of the resulting layer, and the like.

The interference material may be sprayed, cast, coated, or dipped directly to the electroactive surface(s) of the sensor. The dispensing of the interference material may be performed using any known thin film technique. Two, three or more layers of interference material may be formed by the sequential application and curing and/or drying of the casting solution.

The concentration of solids in the casting solution may be adjusted to deposit a sufficient amount of solids or film on the electrode in one layer (e.g., in one dip or spray) to form a layer sufficient to block an interferant with an oxidation or reduction potential otherwise overlapping that of a measured species (e.g., H₂O₂), measured by the sensor. For example, the casting solution's percentage of solids may be adjusted such that only a single layer is required to deposit a sufficient amount to form a functional interference layer that substantially prevents or reduces the equivalent glucose signal of the interferant measured by the sensor. A sufficient amount of interference material would be an amount that substantially prevents or reduces the equivalent glucose signal of the interferant of less than about 30, 20 or 10 mg/dl. By way of example, the interference layer is preferably configured to substantially block about 30 mg/dl of an equivalent glucose signal response that otherwise would be produced by acetaminophen by a sensor without an interference layer. Such equivalent glucose signal response produced by acetaminophen would include a therapeutic dose of acetaminophen. Any number of coatings or layers formed in any order may be suitable for forming the interference layer of the sensor disclosed herein.

In one aspect, the interference layer is deposited either directly onto the electroactive surfaces of the sensor or onto a material or layer in direct contact with the surface of the electrode. Preferably, the interference layer is deposited directly onto the electroactive surfaces of the sensor substantially without an intervening material or layer in direct contact with the surface of the electrode. It has been surprisingly found that configurations comprising the interference layer deposited directly onto the electroactive surface of the sensor substantially eliminates the need for an intervening layer between the electroactive surface and the interference layer while still providing a rapid and accurate signal representative of the analyte.

The interference layer may be applied to provide a thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 1, 1.5 or 2 microns to about 2.5 or 3 microns. Thicker membranes may also be desirable in certain embodiments, but thinner membranes may be generally preferred because they generally have a lower affect on the rate of diffusion of hydrogen peroxide from the enzyme membrane to the electrodes.

### Enzyme Layer

The sensor or sensor assembly disclosed herein includes an enzyme layer. The enzyme layer may be formed a hydrophilic polymer-enzyme composition. It has been surprisingly found that the configuration where the enzyme layer is deposited directly onto at least a portion of the interference layer may substantially eliminate the need for an intervening layer between the interference layer and the enzyme layer while still providing a rapid and accurate signal representative of the analyte. In one aspect, the enzyme layer comprises an enzyme deposited directly onto at least a portion of the interference layer.

In one aspect, the enzyme layer comprises a enzyme and a hydrophilic polymer selected from poly-N-vinylpyrrolidone (PVP), poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polymers with pendent ionizable groups (polyelectrolytes) and copolymers thereof. Preferably, the enzyme layer comprises poly-N-vinylpyrrolidone. Most preferably, the enzyme layer comprises glucose oxidase, poly-N-vinylpyrrolidone and an amount of crosslinking agent sufficient to immobilize the enzyme. The enzyme layer may be as described in co-pending U.S. application Ser. No. 12/199,782, filed Aug. 27, 2009, entitled “Analyte Sensor,” which is hereby incorporated by reference.

The molecular weight of the hydrophilic polymer of the enzyme layer is preferably such that fugitive species are prevented or substantially inhibited from leaving the sensor environment and more particularly, fugitive species are prevented or substantially inhibited from leaving the enzyme's environment when the sensor is initially deployed.

The hydrophilic polymer of the enzyme layer may further include at least one protein and/or natural or synthetic material. For example, the hydrophilic polymer-enzyme composition of the enzyme layer may further include, for example, serum albumins, polyallylamines, polyamines and the like, as well as combination thereof.

The enzyme of the enzyme layer is preferably immobilized in the sensor. The enzyme may be encapsulated within the hydrophilic polymer and may be cross-linked or otherwise immobilized therein. The enzyme may be cross-linked or otherwise immobilized optionally together with at least one protein and/or natural or synthetic material. In one aspect, the hydrophilic polymer-enzyme composition comprises glucose oxidase, bovine serum albumin, and poly-N-vinylpyrrolidone. The composition may further include a cross-linking agent, for example, a dialdehyde such as glutaraldehdye, to cross-link or otherwise immobilize the components of the composition.

In one aspect, other proteins or natural or synthetic materials may be substantially excluded from the hydrophilic polymer-enzyme composition of the enzyme layer. For example, the hydrophilic polymer-enzyme composition may be substantially free of bovine serum albumin. Bovine albumin-free compositions may be desirable for meeting various governmental regulatory requirements. Thus, in one aspect, the enzyme layer comprises glucose oxidase and a sufficient amount of cross-linking agent, for example, a dialdehyde such as glutaraldehdye, to cross-link or otherwise immobilize the enzyme. In other aspect, the enzyme layer comprises glucose oxidase, poly-N-vinylpyrrolidone and a sufficient amount of cross-linking agent to cross-link or otherwise immobilize the enzyme.

The enzyme layer thickness may be from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns. Preferably, the enzyme layer is deposited by spray or dip coating, however, other methods of forming the enzyme layer may be used. The enzyme layer may be formed by dip coating and/or spray coating one or more layers at a predetermined concentration of the coating solution, insertion rate, dwell time, withdrawal rate, and/or desired thickness.

### Flux-Limiting Layer

The sensor or sensor assembly includes a flux-limiting layer covering the subsequent layers described above, where the flux-limiting layer alters or changes the diffusion of one or more of the analytes of interest. Although the following is directed to a flux-limiting layer for an electrochemical glucose sensor, the flux-limiting layer may be modified for other analytes and co-reactants as well.

In one aspect, the flux-limiting layer comprises a semi-permeable material that controls the flux of oxygen and/or glucose to the underlying enzyme layer, preferably providing oxygen in a non-rate-limiting excess. As a result, the upper limit of linearity of glucose measurement is extended to a much higher value than that which is achieved without the flux-limiting layer. In one embodiment, the flux-limiting layer exhibits an oxygen to glucose permeability ratio of from about 50:1 or less to about 400:1 or more, preferably about 200:1. Other flux limiting membranes may be used or combined, such as a membrane with both hydrophilic and hydrophobic polymeric regions, to control the diffusion of analyte and optionally co-analyte to an analyte sensor. For example, a suitable membrane may include a hydrophobic polymer matrix component such as a polyurethane, or polyetherurethaneurea. In one aspect, the material that forms the basis of the hydrophobic matrix of the membrane can be any of those known in the art as appropriate for use as membranes in sensor devices and as having sufficient permeability to allow relevant compounds to pass through it, for example, to allow an oxygen molecule to pass through the membrane from the sample under examination in order to reach the active enzyme or electrochemical electrodes. For example, non-polyurethane type membranes such as vinyl polymers, polyethers, polyesters, polyamides, inorganic polymers such as polysiloxanes and polycarbosiloxanes, natural polymers such as cellulosic and protein based materials, and mixtures or combinations thereof may be used. Preferably, the flux-limiting layer is a dielectric (non-conductive) material. In one aspect, the flux-limiting membrane is selected from vinyl polymers, polysilicones, polyurethanes, or copolymers or blends thereof.

In one aspect, the flux limiting layer comprises a polyethylene oxide component. For example, a hydrophobic-hydrophilic copolymer comprising polyethylene oxide is a polyurethane polymer that includes about 20% hydrophilic polyethylene oxide. The polyethylene oxide portions of the copolymer are thermodynamically driven to separate from the hydrophobic portions (e.g., the urethane portions) of the copolymer and the hydrophobic polymer component. The 20% polyethylene oxide-based soft segment portion of the copolymer used to form the final blend affects the water pick-up and subsequent glucose permeability of the membrane.

In one aspect, the flux limiting membrane substantially excludes condensation polymers such as silicone and urethane polymers and/or copolymers or blends thereof. Such excluded condensation polymers typically contain residual heavy metal catalytic material that may otherwise be toxic if leached and/or difficult to completely remove, thus rendering their use in such sensors undesirable for safety and/or cost.

In another aspect, the material that comprises the flux-limiting layer may be a vinyl polymer appropriate for use in sensor devices having sufficient permeability to allow relevant compounds to pass through it, for example, to allow an oxygen molecule to pass through in order to reach the active enzyme or electrochemical electrodes. Examples of materials which may be used to make the flux-limiting layer include vinyl polymers having vinyl ester monomeric units. In a preferred embodiment, a flux limiting membrane comprises poly ethylene vinyl acetate (EVA polymer). In other aspects, the flux limiting membrane comprises poly(methylmethacrylate-co-butyl methacrylate) blended with the EVA polymer. The EVA polymer or its blends may be cross-linked, for example, with diglycidyl ether. Films of EVA are very elastomeric, which may provide resiliency to the sensor for navigating a tortuous path, for example, into venous anatomy.

The EVA polymer may be provided from a source having a composition of about 40 wt % vinyl acetate (EVA-40). The EVA polymer is preferably dissolved in a solvent for dispensing on the sensor or sensor assembly. The solvent should be chosen for its ability to dissolve EVA polymer, to promote adhesion to the sensor substrate and enzyme electrode, and to form a solution that may be effectively applied (e.g. spray-coated or dip coated). Solvents such as cyclohexanone, paraxylene, and tetrahydrofuran may be suitable for this purpose. The solution may include about 0.5 wt % to about 6.0 wt % of the EVA polymer. In addition, the solvent should be sufficiently volatile to evaporate without undue agitation to prevent issues with the underlying enzyme, but not so volatile as to create problems with the spray process. In a preferred embodiment, the vinyl acetate component of the flux limiting membrane includes about 20% vinyl acetate. In preferred embodiments, the flux limiting membrane is deposited onto the enzyme layer to yield a layer thickness of from about 0.05 microns or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 microns to about 4, 5, 6, 7, 8, 9, 10, 11, 12, 13, 14, 15, 16, 17, 18, 19, or 19.5 microns, and more preferably still from about 5, 5.5 or 6 microns to about 6.5, 7, 7.5 or 8 microns. The flux limiting membrane may be deposited onto the enzyme layer by spray coating or dip coating. In one aspect, the flux limiting membrane is deposited on the enzyme layer by dip coating a solution of from about 1 wt. % to about 5 wt. % EVA polymer and from about 95 wt. % to about 99 wt. % solvent.

In one aspect, an electrochemical analyte sensor is provided comprising a flux limiting membrane covering the enzyme layer, the interference layer and at least a portion of the electroactive surface. Thus, the sensor comprises at least one electroactive surface, an interference layer comprising an interference layer comprising a cellulosic derivative in contact with and at least partially covering at least a portion of the electroactive surface, an enzyme layer comprising a hydrophilic polymer-enzyme composition, at least a portion of the enzyme layer in contact with and at least partially covering the interference layer, and a flux limiting membrane covering the enzyme layer, the interference layer and at least a portion of the electroactive surface.

### Hydrophilic Polymer Membrane

The electrochemical sensor comprises a hydrophilic polymer membrane adjacent to the flux-limiting layer. The hydrophilic polymer membrane may be formed from poly-N-vinylpyrrolidone (PVP), poly-N-vinyl-3-ethyl-2-pyrrolidone, poly-N-vinyl-4,5-dimethyl-2-pyrrolidone, polyacrylamide, poly-N,N-dimethylacrylamide, polyvinyl alcohol, polyvinylacetate, polymers with pendent ionizable groups (polyelectrolytes) and copolymers thereof. Thus, in one aspect, the “hydrophilic polymer membrane” may comprise the same material or a different material as the “hydrophilic layer” described above. In one aspect, the “hydrophilic polymer membrane” comprises the same material as the “hydrophilic layer.”

In one aspect, the hydrophilic polymer membrane is essentially water-insoluble. As used herein, the phase “water-insoluble” refers to a hydrophilic polymer membrane that, when exposed to an excess of water, may swell or otherwise absorb water to an equilibrium volume, but does not dissolve into the aqueous solution. As such, a water-insoluble material generally maintains its original physical structure during the absorption of the water and, thus, must have sufficient physical integrity to resist flow and diffusion away or with its environment. As used herein, a material will be considered to be water insoluble when it substantially resists dissolution in excess water to form a solution, and/or losing its initial, film form and resists becoming essentially molecularly dispersed throughout the water solution. Thus, in one aspect, the hydrophilic polymer membrane will not degrade or diffuse away from the flux-limiting layer during use, for example, during in vivo use.

In one aspect, the hydrophilic polymer membrane is adjacent the flux-limiting layer of the sensor. Generally, the flux-limiting layer is the outermost layer of the sensor, but other chemicals or materials may be present on the flux-limiting layer as well as the hydrophilic polymer membrane. In one aspect, the hydrophilic polymer membrane is coated onto the flux-limiting layer using conventional coating and/or dipping and/or spraying techniques. The thickness of the hydrophilic polymer membrane may be chosen to provide the optimal reduction or elimination of EMF or RF disruption using routine experimental methods provided that the thickness of the hydrophilic membrane does not materially affect other performance requirements of the sensor and/or does not materially affect the ability to introduce the sensor into the host or other device.

In one aspect, the hydrophilic polymer membrane is a polyelectrolyte. Polyelectrolytes are high molecular weight materials having pendent ionizable groups. As electrolytes, polyelectrolytes exhibit the advantageous ionic properties required for stable sensor functioning, such as charge neutralization and charge transfer abilities. Due to their large size, polyelectrolytes substantially reduce or eliminate diffusion of the electrolytic species to the surrounding medium. Thus, a polyelectrolyte may substantially maintain electroneutrality about the sensor and/or reduce or eliminate output signal disruption when exposed to an external EMF or RF source.

In one aspect, the polyelectrolyte may be comprised of polyacids, while other aspects may utilize polybases or polyampholytes as the polyelectrolyte. Further aspects may utilize a polyelectrolyte comprising a polyelectrolyte salt, or polysalt.

In one aspect, the polyelectrolyte comprises pharmaceutically acceptable polysalts. A pharmaceutically acceptable salt is one which is safe and effective for use in humans. For example, pharmaceutically acceptable salts may include polycations with counterions comprising sulfate, pyrosulfate, bisulfate, sulfite, bisulfite, phosphate, monohydrogenphosphate, dihydrogenphosphate, metaphosphate, pyrophosphate, (bi)carbonate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, beta-hydroxybutyrate, glycolate, maleate, tartrate, methanesulfonate, propanesulfonate, naphthalene-1-sulfonate, naphthalene-2-sulfonate, mandelate, or polyanions with positive counterions from elements such as aluminum, calcium, lithium, magnesium, potassium, sodium, and zinc, or from organic compounds such as benzalkonium, pyridinium, quaternary alkyl or arylammonium, or other organic cations, among others.

Generally, polyelectrolytes have numerous ionizable groups, and thus may be highly charged. In one aspect, the polyelectrolyte may be comprised of polyelectrolytes with multiple ionizable groups. In a further aspect, the polyelectrolyte layer may be comprised of highly charged polyelectrolytes without terminal ionizable groups (e.g., Nafion).

In one aspect, the polyelectrolyte may be comprised of a polyelectrolyte comprising sulfonate functionality. Incorporating a polyelectrolyte with sulfonate functionality may be advantageous for analyte sensors, as sulfonate groups are the salts of strong acids and therefore have little influence on the local pH. For example, a polystyrene sulfonate, such as poly(sodium-4-styrene sulfonate), or copolymers of polystyrene sulfonate and maleic acid, such as poly(4-styrene sulfonic acid-co-maleic acid) Na salt, or mixtures thereof may be utilized.

In a further aspect, the polyelectrolyte may be comprised of heparin. Heparin, a naturally occurring polysaccharide polyelectrolyte with sulfonate functionality. In one aspect, benzalkonium heparin is used as the polyelectrolyte. Other salts of heparin may be used, preferably pharmaceutically acceptable salts of heparin. Benzalkonium heparin is frequently used as an anticoagulant on medical devices or used to inhibit blood coagulation in a patient. Thus, one advantage of heparin polyelectrolytes, such as benzalkonium heparin, is that any heparin polyelectrolyte released from the sensor would likely not cause a toxic response in the subject.

In another aspect, the polyelectrolyte may comprise carboxylic acid functionality. Examples of suitable polyelectrolytes with carboxylic acid functionality include polyacrylic acid and polyalkylacrylic acid, where the alkyl is C₁-C₄. In one aspect, the polyelectrolytes with carboxylic acid functionality include polyacrylic acid, polymethacrylic and copolymers or blends thereof.

Any non-toxic polyelectrolyte salt could be utilized as the hydrophilic polymer membrane. One skilled in the art of polymer science can appreciate the very wide diversity of possible combinations of polyions (polymers containing repeat linkages with positive or negative charges) and the associated counterions, and will recognize that the list above is not by any means exhaustive, and other possible combinations are considered to be inclusive, including the possible combination of one or more polyanion and one or more polycation to form a relatively insoluble polyelectrolyte as the hydrophilic polymer membrane.

In one aspect, the hydrophilic membrane is coupled to the flux-limiting layer of the sensor. For example, the hydrophilic membrane may be covalently or ionically attached to the flux-limiting layer of the sensor. By way of example, functional groups of the flux-limiting layer may covalently or ionically couple all or part of the hydrophilic membrane. Alternatively, the flux-limiting layer may be chemically modified to covalently or ionically couple all or part of the hydrophilic membrane. Chemical modification of the flux-limiting layer may include gas plasma treatment or chemical reduction/oxidation processes. Covalent coupling with the hydrophilic membrane may employ coupling agents know in the art, such as 1-ethyl-3(3-dimethyl aminopropyl)carbodiimide hydrochloride (EDC) or N-hydroxysuccinimide or other water-soluble carbodiimides, and may be employed with enhancers such as N-hydroxysulfosuccinimide (sulfa-NHS), although other suitable enhancers, such as N-hydroxysuccinimide (NHS), can alternatively be used.

The hydrophilic membrane when disposed over the flux-limiting layer of the biosensor reduces or eliminates disruption of the output signal generated by the sensor. Typically, the electrochemical sensor output signal is a current proportional to the concentration of a target analyte being measured. In an in vivo environment, such a target analyte may be glucose. During use of the sensor the output signal is received by a control unit which converts the signal to a analyte concentration value. In the event of an external EMF or RF source of sufficient strength and duration in the proximity of the sensor, the output signal may spike and/or flat-line or otherwise fail to accurately represent the target analyte concentration in the absence of the hydrophilic membrane disposed over the flux-limiting layer. Thus, the use of the hydrophilic polymer membrane may reduce spiking and/or flat-lining of the sensor output and may further provide for the output signal to resume or return to accurately represent the target analyte concentration.

### Bioactive Agents

In some alternative embodiments, a bioactive agent may be optionally incorporated into the above described sensor system, such that the bioactive diffuses out into the biological environment adjacent to the sensor. Additionally or alternately, a bioactive agent may be administered locally at the exit-site or implantation-site. Suitable bioactive agents include those that modify the subject's tissue response to any of the sensor or components thereof. For example, bioactive agents may be selected from anti-inflammatory agents, anti-infective agents, anesthetics, inflammatory agents, growth factors, immunosuppressive agents, antiplatelet agents, anti-coagulants, anti-proliferates, ACE inhibitors, cytotoxic agents, anti-barrier cell compounds, vascularization-inducing compounds, anti-sense molecules, or mixtures thereof.

### Flexible Substrate Sensor Assembly Adapted for Intravenous Insertion

In one aspect, an electrochemical analyte sensor assembly may be configured for an intravenous insertion to a vascular system of a subject. In order to accommodate the sensor within the confined space of a device suitable for intravenous insertion, the sensor assembly may comprise a flexible substrate, such as a flex circuit. For example, the flexible substrate of the flex circuit may be configured as a thin conductive electrodes coated on a non-conductive material such as a thermoplastic or thermoset. Conductive traces may be formed on the non-conductive material and electrically coupled to the thin conductive electrodes. The electrodes of the flex circuit may be as described above.

The flex circuit may comprise at least one reference electrode and at least one working electrode, the at least one working electrode having an electroactive surface capable of providing a detectable electrical output upon interaction with an electrochemically detectable species. The flex circuit may further comprise at least one counter electrode. In one aspect, the flex circuit contains two or more working electrodes and two or more counter electrodes. In one aspect, the flex circuit contains two or more working electrodes, two or more blank electrodes and two or more counter electrodes.

An interference layer comprising a cellulosic derivative may be placed in direct contact with and at least partially covering a portion of the electroactive surface of working electrode of the flex circuit. An enzyme layer comprising a hydrophilic polymer-enzyme composition capable of enzymatically interacting with an analyte so as to provide the electrochemically detectable species, may be placed such that at least a portion thereof is in direct contact with and at least partially covering the interference layer. A membrane, such as a membrane that alters the flux of an analyte of interest may be placed such that it covers the hydrophilic polymeric layer, the interference layer and at least a portion of the electroactive surface of the flex circuit. The flex circuit preferably is configured to be electrically configurable to a control unit. An example of an electrode of a flex circuit and it construction is found in co-assigned U.S. Application Nos. 2007/0202672 and 2007/0200254, incorporated herein by reference in their entirety.

Medical devices adaptable to the sensor assembly as described above include, but are not limited to a central venous catheter (CVC), a pulmonary artery catheter (PAC), a probe for insertion through a CVC or PAC or through a peripheral IV catheter, a peripherally inserted catheter (PICC), Swan-Ganz catheter, an introducer or an attachment to a Venous Arterial blood Management Protection (VAMP) system. Any size/type of Central Venous Catheter (CVC) or intravenous devices may be used or adapted for use with the sensor assembly.

For the foregoing discussion, the implementation of the sensor or sensor assembly is disclosed as being placed within a catheter, however, other devices as described above are envisaged and incorporated in aspects of the embodiments disclosed herein. The sensor assembly will preferably be applied to the catheter so as to be flush with the OD of the catheter tubing. This may be accomplished, for example, by thermally deforming the OD of the tubing to provide a recess for the sensor. The sensor assembly may be bonded in place, and sealed with an adhesive (ie. urethane, 2-part epoxy, acrylic, etc.) that will resist bending/peeling, and adhere to the urethane CVC tubing, as well as the materials of the sensor. Small diameter electrical wires may be attached to the sensor assembly by soldering, resistance welding, or conductive epoxy. These wires may travel from the proximal end of the sensor, through one of the catheter lumens, and then to the proximal end of the catheter. At this point, the wires may be soldered to an electrical connector.

The sensor assembly as disclosed herein can be added to a catheter in a variety of ways. For example, an opening may be provided in the catheter body and a sensor or sensor assembly may be mounted inside the lumen at the opening so that the sensor would have direct blood contact. In one aspect, the sensor or sensor assembly may be positioned proximal to all the infusion ports of the catheter. In this configuration, the sensor would be prevented from or minimized in measuring otherwise detectable infusate concentration instead of the blood concentration of the analyte. Another aspect, an attachment method may be an indentation on the outside of the catheter body and to secure the sensor inside the indentation. This may have the added advantage of partially isolating the sensor from the temperature effects of any added infusate. Each end of the recess may have a skived opening to 1) secure the distal end of the sensor and 2) allow the lumen to carry the sensor wires to the connector at the proximal end of the catheter.

Preferably, the location of the sensor assembly in the catheter will be proximal (upstream) of any infusion ports to prevent or minimize IV solutions from affecting analyte measurements. In one aspect, the sensor assembly may be about 2.0 mm or more proximal to any of the infusion ports of the catheter.

In another aspect, the sensor assembly may be configured such that flushing of the catheter (ie. saline solution) may be employed in order to allow the sensor assembly to be cleared of any material that may interfere with its function.

### Sterilization of the Sensor or Sensor Assembly

Generally, the sensor or the sensor assembly as well as the device that the sensor is adapted to are sterilized before use, for example, in a subject. Sterilization may be achieved using radiation (e.g., electron beam or gamma radiation), ethylene oxide or flash-UV sterilization, or other means know in the art.

Disposable portions, if any, of the sensor, sensor assembly or devices adapted to receive and contain the sensor preferably will be sterilized, for example using e-beam or gamma radiation or other know methods. The fully assembled device or any of the disposable components may be packaged inside a sealed non-breathable container or pouch.

Central line catheters may be known in the art and typically used in the Intensive Care Unit (ICU)/Emergency Room of a hospital to deliver medications through one or more lumens of the catheter to the patient (different lumens for different medications). A central line catheter is typically connected to an infusion device (e.g. infusion pump, IV drip, or syringe port) on one end and the other end inserted in one of the main arteries or veins near the patient's heart to deliver the medications. The infusion device delivers medications, such as, but not limited to, saline, drugs, vitamins, medication, proteins, peptides, insulin, neural transmitters, or the like, as needed to the patient. In alternative embodiments, the central line catheter may be used in any body space or vessel such as intraperitoneal areas, lymph glands, the subcutaneous, the lungs, the digestive tract, or the like and may determine the analyte or therapy in body fluids other than blood. The central line catheter may be a double lumen catheter. In one aspect, an analyte sensor is built into one lumen of a central line catheter and is used for determining characteristic levels in the blood and/or bodily fluids of the user. However, it will be recognized that further embodiments may be used to determine the levels of other agents, characteristics or compositions, such as hormones, cholesterol, medications, concentrations, viral loads (e.g., HIV), or the like. Therefore, although aspects disclosed herein may be primarily described in the context of glucose sensors used in the treatment of diabetes/diabetic symptoms, the aspects disclosed may be applicable to a wide variety of patient treatment programs where a physiological characteristic is monitored in an ICU, including but not limited to blood gases, pH, temperature and other analytes of interest in the vascular system.

In another aspect, a method of intravenously measuring an analyte in a subject is provided. The method comprises providing a catheter comprising the sensor assembly as described herein and introducing the catheter into the vascular system of a subject. The method further comprises measuring an analyte.

Accordingly, sensors and methods have been disclosed and described for reducing or eliminating disruption of the output signal of an electrochemical sensor when used in the presence of an EMF or RF source, such as an ESU.

Referring now to the Figures, FIG. 1 is an amperometric sensor 11 in the form of a flex circuit that incorporates a sensor embodiment of the invention. The sensor or sensors 11 may be formed on a substrate 13 (e.g., a flex substrate, such as copper foil laminated with polyimide). One or more electrodes 15, 17, and 19 may be attached or bonded to a surface of the substrate 13. The sensor 11 is shown with a reference electrode 15, a counter electrode 17, and a working electrode 19. In another embodiment, one or more additional working electrodes may be included on the substrate 13. Electrical wires 210 may transmit power to the electrodes for sustaining an oxidation or reduction reaction, and may also carry signal currents to a detection circuit (not shown) indicative of a parameter being measured. The parameter being measured may be any analyte of interest that occurs in, or may be derived from, blood chemistry. In one embodiment, the analyte of interest may be hydrogen peroxide, formed from reaction of glucose with glucose oxidase, thus having a concentration that is proportional to blood glucose concentration.

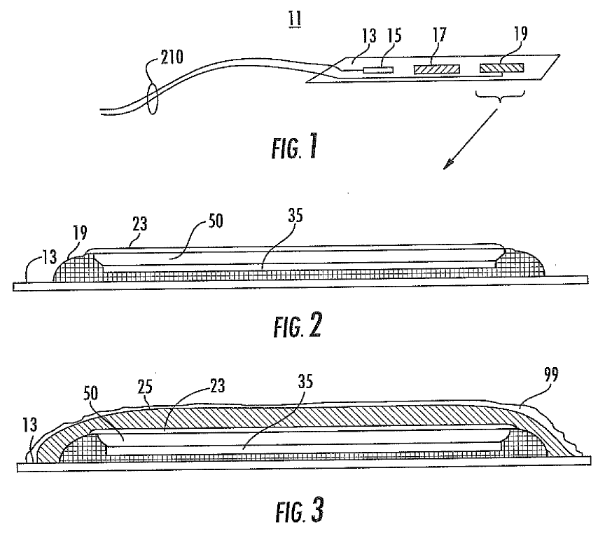

FIG. 2 depicts a cross-sectional side view of a portion of substrate 13 in the vicinity of working electrode 19 of an embodiment of the invention. Working electrode 19 may be at least partially coated with hydrophilic layer 35. Hydrophilic layer 35 may be at least partially coated with interference layer 50. Interference layer 50 may be at least partially coated with an enzyme layer 23, the enzyme layer selected to chemically react when the sensor is exposed to certain reactants, for example, those found in the bloodstream. For example, in an embodiment for a glucose sensor, enzyme layer 23 may contain glucose oxidase, such as may be derived from Aspergillus niger (EC 1.1.3.4), type II or type VII.

FIG. 3 shows a cross sectional side view of the working electrode site on the sensor substrate 13 further comprising flux-limiting layer 25 covering enzyme layer 23, interference layer 50, hydrophilic layer 35 and at least a portion of electrode 19. Flux-limiting layer 25 may selectively allow diffusion, from blood to the enzyme layer 23, of a blood component that reacts with the enzyme. In a glucose sensor embodiment, the membrane 25 passes an abundance of oxygen, and selectively limits glucose, to the enzyme layer 23. In addition, the flux-limiting layer 25 may have adhesive properties that may mechanically seal the enzyme layer 23 to the sub-layers and/or working electrode 19, and may also seal the working electrode 19 to the sensor substrate 13. It is herein disclosed that a flux-limiting layer 25 may serve as a flux limiter, but also serve as a sealant or encapsulant at the enzyme/electrode boundary and at the electrode/substrate boundary. Hydrophilic polymer membrane 99 is shown covering the flux-limiting layer to provide reduction of signal output disruption when sensor is used in the presence of an external EMF or RF field.

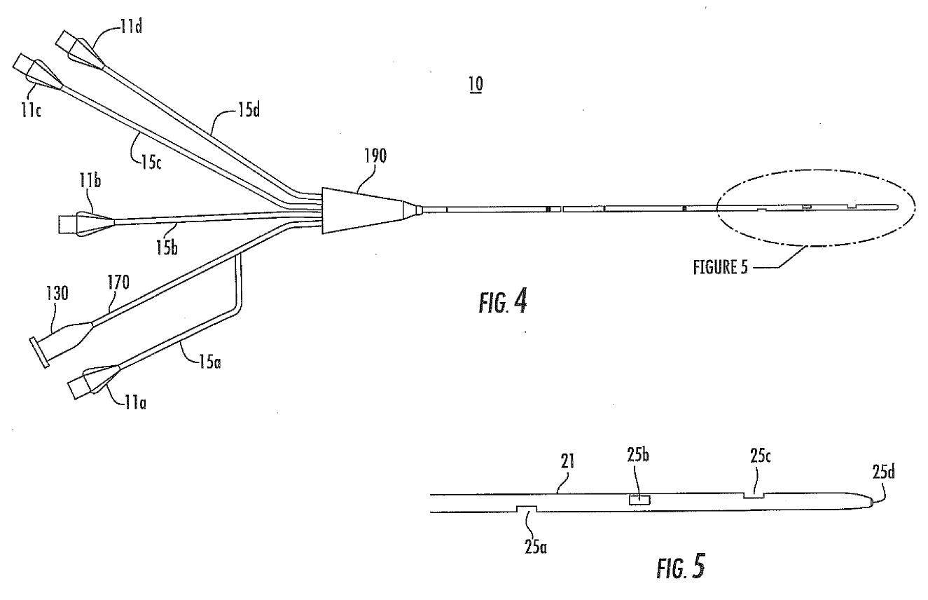

Referring now to FIGS. 4-5, aspects of the sensor adapted to a central line catheter with a sensor or sensor assembly are discussed as exemplary embodiments, without limitation to any particular intravenous device. FIG. 4 shows a sensor assembly within a multilumen catheter. The catheter assembly 10 may include multiple infusion ports 11a, 11b, 11c, 11d and one or more electrical connectors 130 at its most proximal end. A lumen 15a, 15b, 15c or 15d may connect each infusion port 11a, 11b, 11c, or 11d, respectively, to a junction 190. Similarly, the conduit 170 may connect an electrical connector 130 to the junction 190, and may terminate at junction 190, or at one of the lumens 15a-15d (as shown). Although the particular embodiment shown in FIG. 4 is a multilumen catheter having four lumens and one electrical connector, other embodiments having other combinations of lumens and connectors are possible within the scope of the invention, including a single lumen catheter, a catheter having multiple electrical connectors, etc. In another embodiment, one of the lumens and the electrical connector may be reserved for a probe or other sensor mounting device, or one of the lumens may be open at its proximal end and designated for insertion of the probe or sensor mounting device.

The distal end of the catheter assembly 10 is shown in greater detail in FIG. 5. At one or more intermediate locations along the distal end, the tube 21 may define one or more ports formed through its outer wall. These may include the intermediate ports 25a, 25b, and 25c, and an end port 25d that may be formed at the distal tip of tube 21. Each port 25a-25d may correspond respectively to one of the lumens 15a-15d. That is, each lumen may define an independent channel extending from one of the infusion ports 11a-11d to one of the tube ports 25a-25d. The sensor assembly may be presented to the sensing environment via positioning at one or more of the ports to provide contact with the medium to be analyzed. In one aspect, the hydrophilic polymer membrane may be coated on outside surface of the catheter.

In another aspect, a method of intravenously measuring an analyte in a subject is provided. The method comprises providing a catheter comprising the sensor assembly as described herein and introducing the catheter into the vascular system of a subject. The method further comprises measuring an analyte.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification may be to be understood as being modified in all instances by the term “about.” Accordingly, unless indicated to the contrary, the numerical parameters set forth herein may be approximations that may vary depending upon the desired properties sought to be obtained.

The above description discloses several methods and materials. These descriptions are susceptible to modifications in the methods and materials, as well as alterations in the fabrication methods and equipment. Such modifications will become apparent to those skilled in the art from a consideration of this disclosure or practice of the disclosure. Consequently, it is not intended that this disclosure be limited to the specific embodiments disclosed herein, but that it cover all modifications and alternatives coming within the true scope and spirit of the claims.

We claim:

1. A method comprising:
providing an in vivo electrochemical biosensor, the biosensor comprising:
an electrode surface;
a flux-limiting layer covering at least a portion of the electrode surface;
covering at least a portion of the flux-limiting layer with a hydrophilic polymer membrane; and
reducing disruption of the output signal of the electrochemical biosensor by an external EMF or external RF source during in vivo use of the biosensor.

2. The method of claim 1, wherein the external EMF or external RF source is generated by an electrosurgical unit (ESU).

3. The method of claim 2, wherein the electrosurgical unit is monopolar or bipolar.

4. The method of claim 2, wherein the electrosurgical unit operates at a frequency between about 350 KHz and about 4 MHz.

5. The method of claim 1, wherein the hydrophilic polymer membrane accelerates reformation of a boundary layer comprising charged species about the flux-limiting layer of the electrochemical biosensor during in vivo use thereof in a subject.

6. The method of claim 1, wherein the hydrophilic polymer membrane is covalently or ionically coupled to the flux-limiting layer.

7. The method of claim 1, wherein the hydrophilic polymer membrane comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyvinyl acetate, polyelectrolyte, and copolymers thereof.

8. The method of claim 1, wherein the hydrophilic polymer membrane is essentially water-insoluble.

9. The method of claim 1, wherein the flux-limiting membrane is selected from the group consisting of vinyl polymers, polysilicones, polyurethanes, and copolymers or blends thereof.

10. The method of claim 9, wherein the flux-limiting membrane is polyethylene vinylacetate.

11. The method of claim 1, the electrochemical sensor further comprising at least one of:
a hydrophilic layer at least partially covering the electrode surface; or
an interference layer at least partially covering the electrode surface; or
an enzyme layer at least partially covering an interference layer.

12. The method of claim 11, wherein the interference layer comprises a cellulosic derivative or cellulose acetate butyrate, or wherein the enzyme layer comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyelectrolyte and copolymers thereof.

13. The method of claim 11, wherein the enzyme layer comprises glucose oxidase, poly-N-vinylpyrrolidone, and optionally an amount of crosslinking agent sufficient to immobilize the glucose oxidase.

14. An electrochemical analyte sensor, comprising:
an in vivo biosensor capable of sensing an analyte level in blood and outputting a signal corresponding to the analyte concentration, the in vivo biosensor comprising:
an electrode surface;
an enzyme layer covering at least a portion of the electrode surface;
a flux-limiting layer covering at least a portion of the enzyme layer and at least a portion of the electrode surface; and
a hydrophilic polymer membrane covering at least a portion of the flux-limiting layer;
wherein disruption of the output signal of the analyte sensor, when operated in the presence of an external EMF or external RF source during in vivo use is reduced.

15. The electrochemical analyte sensor of claim 14, wherein the hydrophilic polymer membrane accelerates reformation of a boundary layer comprising charged species about the flux-limiting layer of the electrochemical biosensor during in vivo use thereof in a subject.

16. The electrochemical analyte sensor of claim 14, wherein the hydrophilic polymer membrane comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyvinyl acetate, polyelectrolyte, and copolymers thereof.

17. The electrochemical analyte sensor of claim 14, wherein the hydrophilic polymer membrane is covalently or ionically coupled to the flux-limiting layer.

18. The electrochemical analyte sensor of claim 14, wherein the hydrophilic polymer membrane is essentially water-insoluble.

19. The electrochemical analyte sensor of claim 14, wherein the flux-limiting membrane is selected from the group consisting of vinyl polymers, polysilicones, polyurethanes, and copolymers or blends thereof.

20. The electrochemical analyte sensor of claim 14, wherein the flux-limiting membrane is poly(ethylene-vinylacetate).

21. The electrochemical analyte sensor of claim 14, further comprising at least one of:
a hydrophilic layer at least partially covering the electrode surface; or
an interference layer at least partially covering the electrode surface; or
an enzyme layer at least partially covering an interference layer.

22. The electrochemical analyte sensor of claim 21, wherein the interference layer comprises a cellulosic derivative or cellulose acetate butyrate, or wherein the enzyme layer comprises a material selected from the group consisting of poly-N-vinylpyrrolidone, poly-N-vinyl-3-ethyl-2-pyrrolidone, poly-N-vinyl-4,5-dimethyl-2-pyrrolidone, polyvinylimidazole, poly-N—N-dimethylacrylamide, polyacrylamide, polyvinyl alcohol, polyethylene glycol, polyelectrolyte, and copolymers thereof.

23. The electrochemical analyte sensor of claim 21, wherein the enzyme layer comprises glucose oxidase, poly-N-vinylpyrrolidone, and optionally an amount of crosslinking agent sufficient to immobilize the glucose oxidase.

24. A method comprising:
providing an in vivo electrochemical biosensor, the biosensor comprising:
an electrode surface;
a hydrophilic layer covering at least a portion of the electrode surface;
an interference layer covering at least a part of the hydrophilic layer;
an enzyme layer covering at least a part of the interference layer;
a flux-limiting layer covering at least a portion of the enzyme layer;
covering at least a portion of the flux-limiting layer with a hydrophilic polymer membrane, wherein the hydrophilic polymer membrane comprises a material selected from the group consisting of poly-N-vinylpyrrolidone, polyacrylamide, polyvinyl acetate, and polyelectrolyte; and
reducing disruption of the output signal of the electrochemical biosensor by an electrosurgical unit (ESU) during in vivo use of the biosensor in a subject.

25. The method of claim 24, wherein the flux-limiting membrane is polyethylene vinylacetate.
